(19) United States
(12) Patent Application Publication
(10) Pub. No.: US 2024/0186013 A1
(43) Pub. Date: Jun. 6, 2024

(54) INFECTION RISK ESTIMATION APPARATUS, INFECTION RISK ESTIMATION METHOD, AND NON-TRANSITORY STORAGE MEDIUM

(71) Applicant: NEC Corporation, Minato-ku, Tokyo (JP)

(72) Inventors: Shihono Mochizuki, Tokyo (JP); Yohei Itou, Tokyo (JP)

(73) Assignee: NEC Corporation, Minato-ku, Tokyo (JP)

(21) Appl. No.: 18/283,237

(22) PCT Filed: Jan. 17, 2022
(86) PCT No.: PCT/JP2022/001370

§ 371 Date: Sep. 21, 2023

(30) Foreign Application Priority Data
Mar. 24, 2021 (JP) 2021-050714

(51) Int. Cl. G16H 50/30; G16H 50/80

(52) U.S. Cl. CPC G16H 50/30; G16H 50/80

(57) ABSTRACT

An infection risk estimation apparatus (100) includes a first acquisition unit (102a) that acquires visitor information including an attribute of a visitor to a target place, a second acquisition unit (102b) that acquires infection status information including infection status of an infection disease in each area, and an estimation unit (104) that acquires, by use of the visitor information and the infection status information, a first risk index R1 indicating a degree of a risk of being infected with the infection disease in the target place.

### TECHNICAL FIELD

The present invention relates to an infection risk estimation apparatus, an infection risk estimation method, and a program.

### BACKGROUND ART

Various techniques for estimating an infection risk of being infected with an infection disease have been suggested.

For example, an information provision method to be executed by a computer of an information provision system for providing information relating to an infection disease described in PTL 1 is disclosed. According to PTL 1, a computer acquires areal infection information from one or more sound recognition apparatuses connected via a network, and computes, based on the acquired areal infection information, an infection risk value representing magnitude of an infection risk of each of the one or more areas. Then, the computer generates, regarding each of the one or more areas, output information according to the computed infection risk value, and transmits, regarding each of the one or more areas, the generated output information to equipment existing in an area being associated with the output information, via the network. According to the description in PTL 1, the areal infection information indicates one or more infection caution levels acquired by analyzing a sound signal by the one or more sound recognition apparatuses, and one or more areas being related to the one or more infection caution levels.

### RELATED DOCUMENT

### Patent Document

PTL 1: Japanese Patent Application Publication No. 2020-27610

### SUMMARY OF THE INVENTION

### Technical Problem

In the information provision method described in PTL 1, an infection risk value is computed based on areal infection information acquired from one or more sound recognition apparatuses. However, it is considered that an infection disease may spread with movement of a person, and it is desirable to be able to learn, for example, a risk of being infected with an infection disease in various places, in order to take an appropriate measure for preventing an infection spread of an infection disease.

The present invention has been made in view of the above circumstances, and one of objects thereof is to support an appropriate measure for preventing an infection spread of an infection disease.

### Solution to Problem

In order to achieve the above object, an infection risk estimation apparatus according to a first aspect of the present invention includes:
an acquisition means for acquiring visitor information including an attribute of a visitor to a target place, and infection status information including infection status of an infection disease in each area; and
an estimation means for acquiring, by use of the visitor information and the infection status information, a first risk index indicating a degree of a risk of being infected with the infection disease in the target place.

In order to achieve the above object, an infection risk estimation method according to a second aspect of the present invention includes,
by a computer:
acquiring visitor information including an attribute of a visitor to a target place, and infection status information including infection status of an infection disease in each area; and
acquiring, by use of the visitor information and the infection status information, a first risk index indicating a degree of a risk of being infected with the infection disease in the target place.

In order to achieve the above object, a program according to a third aspect of the present invention is a program for causing a computer to execute:
acquiring visitor information including an attribute of a visitor to a target place, and infection status information including infection status of an infection disease in each area; and
acquiring, by use of the visitor information and the infection status information, a first risk index indicating a degree of a risk of being infected with the infection disease in the target place.

### Advantageous Effects of Invention

The present invention enables supporting an appropriate measure for preventing an infection spread of an infection disease.

### BRIEF DESCRIPTION OF THE DRAWINGS

FIG. 1 is a diagram illustrating one example of a functional configuration of an infection risk estimation apparatus according to a first example embodiment of the present invention.

FIG. 2 is a diagram illustrating one example of visitor information.

FIG. 3 is a diagram illustrating one example of age group-facility type information.

FIG. 4 is a diagram illustrating one example of areal facility information.

FIG. 5 is a diagram illustrating one example of target place risk information.

FIG. 6 is a diagram illustrating one example of output information including occurrence place information and diffusion place information.

FIG. 7 is a diagram illustrating one example of caution facility information.

FIG. 8 is a diagram illustrating one example of a physical configuration of the infection risk estimation apparatus according to the first example embodiment of the present invention.

FIG. 9 is a flowchart illustrating one example of infection risk estimation processing according to the first example embodiment of the present invention.

FIG. 10 is a diagram illustrating one example of a functional configuration of an infection risk estimation apparatus according to a second example embodiment of the present invention.

FIG. 11 is a flowchart illustrating one example of infection risk estimation processing according to the second example embodiment of the present invention.

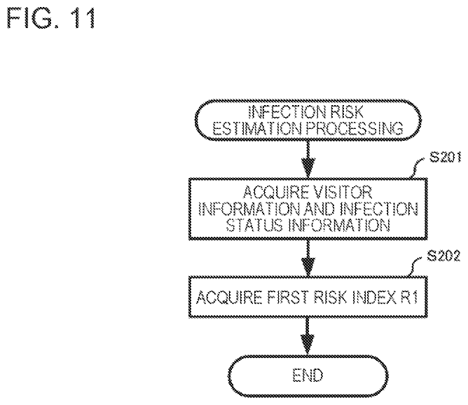

### DESCRIPTION OF EMBODIMENTS

Hereinafter, example embodiments of the present invention are described by use of the drawings. Note that, a similar reference sign is assigned to a similar component in all the drawings, and description is omitted as appropriate.

### First Example Embodiment

An infection risk estimation apparatus 100 according to a first example embodiment of the present invention is an apparatus that supports spread prevention of an infection disease by acquiring a first risk index R1 indicating a degree of a risk of being infected with an infection disease in a plurality of target places.

A target place is a place where there is a possibility that a person visits from various places, and is a tourist spot, a facility, or the like. As a tourist spot, for example, a shrine, a temple, and a famous place can be cited. Moreover, as a facility, for example, a landmark, a theme park, a park, a service area, a movie theater, an art gallery, a museum, a theater, an exhibition hall, a stadium, a ball game ground, a gymnasium, a golf course, an amusement park, and a shopping mall can be cited. Note that, a target place is not limited thereto, and one or more may be determined as appropriate.

An infection disease is, but is not limited to, for example, influenza or COVID-19.

### Functional Configuration of Infection Risk Estimation Apparatus 100

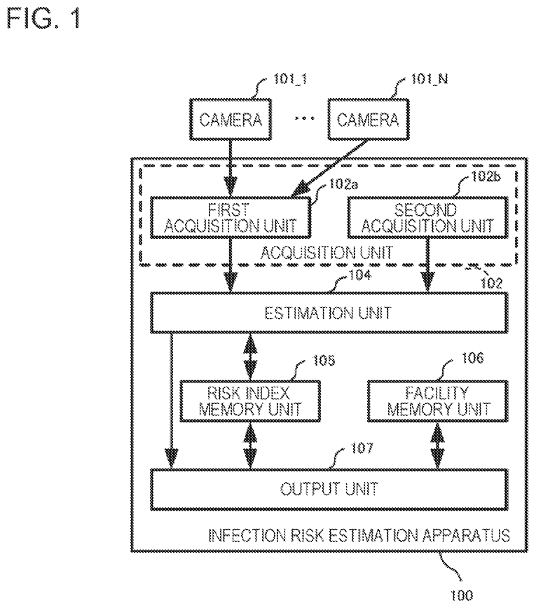

The infection risk estimation apparatus 100 according to the present example embodiment includes, as a functional configuration thereof is illustrated in FIG. 1, cameras 101_1 to 101_N (N: an integer equal to or more than 1), an acquisition unit 102 including a first acquisition unit 102a and a second acquisition unit 102b, an estimation unit 104, a risk index memory unit 105, a facility memory unit 106, and an output unit 107.

Each of the cameras 101_1 to 101_N is a camera installed in a parking lot or a target place. Each of the cameras 101_1 to 101_N is connected to the infection risk estimation apparatus 100 via a network in such a way as to be able to transmit and receive information to and from each other, and the infection risk estimation apparatus 100 acquires generated transportation facility information.

When not particularly distinguished, the cameras 101_1to 101_N are also simply referred to as a “camera 101” below.

The camera 101 installed in a parking lot is provided, for example, at an entrance of the parking lot, captures an image of a license plate of a car parked in the parking lot, and generates image information including the captured image of the license plate. The camera 101 installed in a target place captures an image of a visitor to the target place, and generates visitor image information indicating the captured image of the visitor.

Each of the cameras 101 installed in a parking lot is associated with the target place based on a previously determined association condition. The association condition includes, for example, being installed in a parking lot within a previously determined distance from a target place, a parking lot as a target place being attached to the target place, and the like.

Herein, when a road near a target place is congested, a parking lot near the target place may become full, and a visitor to the target place may utilize a parking lot farther than when the road is not congested. Thus, when a road near a target place is congested for equal to or more than a predetermined time (e.g., one hour), an association condition may be changed depending on whether the road near the target place is congested, in such a way that a parking lot farther than when the road is not congested is associated.

The first acquisition unit 102a acquires visitor information 110. Moreover, the first acquisition unit 102a acquires wearing rate information indicating a wearing rate indicating a percentage of a person wearing a mask as a cover covering a mouth among visitors.

The visitor information 110 is information relating to a visitor to a target place, and includes an attribute of a visitor, such as a residential area Ai of the visitor, and an age group of the visitor.

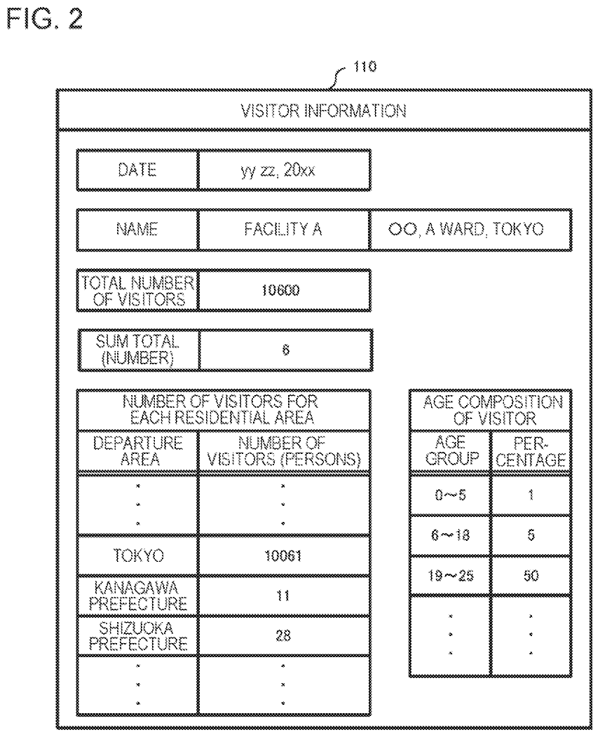

As illustrated in FIG. 2, the visitor information 110 according to the present example embodiment includes date information, target place information, a total number of visitors to a target place, a sum total SN, and the number of visitors and an age composition for each residential area as an attribute of a visitor.

The date information indicates a date. The target place information is information for identifying the target place, and includes, for example, a name and an address indicating a location.

The total number of visitors to a target place indicates a total number of visitors coming to the target place on a date indicated by the date information. A total number of visitors counted at an entrance of a target place is input by a user, and thereby acquired by the first acquisition unit 102a. Note that, the first acquisition unit 102a may count and thereby acquire the number of persons determined by use of a conventional image processing technique.

The sum total indicates a sum total of cars parked in a parking lot being associated with a target place on a date indicated by the date information.

The number of visitors for each residential area indicates the number of visitors for each residential area of a visitor coming to a target place on a date indicated by the date information. The residential area Ai according to the present example embodiment is each prefecture, and i is an integer of 1 to 47. Note that, a residential area is not limited to a prefecture, and may be an appropriately determined region such as a region corresponding to a place name indicating a base of use of a car (i.e., a jurisdiction of a car).

The age composition indicates an age composition of a visitor coming to a target place on a date indicated by date information, and is, for example, a percentage of visitors for each age group to a total number of visitors. Note that, the age composition may be the number of visitors for each age group of visitors coming to a target place on a date indicated by the date information.

Specifically, the first acquisition unit 102a acquires transportation facility information and past visitor information in a target place in order to acquire the number of visitors and an age composition for each residential area.

The transportation facility information is information acquired in a parking lot as a transportation facility, and is information acquired in a previously associated parking lot being previously associated with a target place by an association condition.

The transportation facility information according to the present example embodiment includes image information indicating an image captured by the camera 101 installed in a parking lot. The first acquisition unit 102a acquires the number of visitors for each residential area by estimating the number of visitors for each residential area, based on the image information included in the transportation facility information.

Past visitor information in a target place includes an age composition of a past visitor as an attribute of a past visitor. Thus, the first acquisition unit 102a acquires an age composition of a past visitor included in past visitor information in a target place, as an age composition of a visitor in the target place.

The place attribute information is information indicating an attribute of a target place. As an example of an attribute of a target place, a type of being an indoor facility or an outdoor facility, and a type of being a facility where density of persons becomes equal to or more than previously determined density or not can be cited.

Further, the first acquisition unit 102a acquires visitor image information from the camera 101, and acquires wearing rate information in a target place by performing image processing on the visitor image information. The image processing uses, for example, a learned learning model that inputs an image of a person and outputs information indicating whether the person is wearing a mask. The first acquisition unit 102a acquires wearing rate information in a target place by deriving a percentage of persons wearing masks among persons included in the visitor image information.

The second acquisition unit 102b acquires infection status information including the infection status of an infection disease in each of the areas Ai.

The infection status of an infection disease in each of the areas Ai is, for example, an infection rate of an infection disease in each prefecture. The infection rate of an infection disease in each prefecture can be acquired, for example, by dividing a daily number of newly infected persons announced in each prefecture by population of a corresponding prefecture.

Note that, the infection status of an infection disease in each area is not limited thereto, and may be, for example, an infection rate of an infection disease in each prefecture, or the like, or may be an infection rate for each province including the whole country or a plurality of prefectures. The infection rate of an infection disease may be a moving average of an infection rate for a previously determined period (e.g., two weeks).

The estimation unit 104 acquires the first risk index R1 by use of visitor information, wearing rate information, and infection status information acquired by each of the first acquisition unit 102a and the second acquisition unit 102b. Then, the estimation unit 104 further acquires, by use of the first risk index R1 and the visitor information, a second risk index R2 and a third risk index R3 being indices according to the number of visitors for each residential area.

Further, the estimation unit 104 determines whether the first risk index R1, the second risk index R2, and the third risk index R3 satisfy a previously determined criterion.

More specifically, the first risk index R1 is, as described above, an index indicating a degree of a risk of being infected with an infection disease in a target place.

The first risk index R1 according to the present example embodiment is derived by deriving a product of the number of visitors and an infection rate for each of the residential areas Ai, and multiplying, by coefficients p to r, a value in which the product is added to each of all the residential areas Ai, for example, as illustrated in Equation (1).

[Mathematical 1]

First risk index R1=Σ(number of visitors to area Ai×infection rate in area Ai)×p×q×r Equation (1)

The area Ai in Equation (1) is a prefecture as described above, and the first risk index R1 in this case is derived by multiplying, by coefficients p to r, a value in which a product of an estimate number of visitors and an infection rate for each prefecture is added in 47 prefectures.

The coefficients p to r are values determined according to each of place attribute information and a wearing rate.

A value appropriately determined according to whether a target place is an outdoor facility clothing or an indoor facility is set for the coefficient p. Generally, in an outdoor facility, ventilation is better than in an indoor facility, and, therefore, it may be difficult for an infection disease to be infected. In such a case, a smaller value may be set for the coefficient p for an outdoor facility than the coefficient p for an indoor facility.

A value appropriately determined according to density of persons in a target place is set for the coefficient q. Generally, it may be difficult to be infected with an infection disease in a facility with high density of persons than in a facility with low density of persons. In such a case, the coefficient q for high density may be set to a larger value than the coefficient q for low density.

A value appropriately determined according to wearing rate information is set for the coefficient r. Generally, it may be more difficult to be infected with an infection disease as a percentage of a person wearing a mask is larger. In such a case, a value previously determined in such a way as to become smaller as a value of wearing rate information is larger may be set for the coefficient r.

The second risk index R2 is an index according to the number of visitors for each residential area regarding a visitor to a target place, and, for example, an index indicating a degree of the number of visitors for each residential area with respect to a total number of visitors is adopted.

Specifically, for example, the second risk index R2 is a percentage of visitors for each residential area, and is acquired by dividing the number of visitors for each residential area by a total number of visitors. Note that, the second risk index R2 may be the number of visitors for each residential area.

The third risk index R3 is an index according to the number of visitors for each age group regarding a visitor to a target place, and, for example, a value indicating a degree of the number of visitors for each age group with respect to a total number of visitors to the target place.

Specifically, for example, the third risk index R3 is a percentage of a visitor for each age group. Note that, the third risk index R3 may be the number of visitors for each age group.

Note that, the third risk index R3 may be an index indicating a degree of the number of visitors for each age group, and may be, for example, the very number of visitors for each residential area regarding a visitor to a target place.

Moreover, the following first to third criteria are previously retained as criteria used by the estimation unit 104 for discrimination.

The first criterion is a criterion for discriminating whether a risk of being infected with an infection disease in a target place is high. The first criterion is previously determined in relation to the first risk index R1 in such a way as, for example, “the first risk index R1 is equal to or more than a first threshold value (e.g., ‘0.5’)”.

The second criterion is a criterion for determining whether a risk that an infection disease is diffused from a target place to the residential area Ai of a visitor is high. The second criterion is previously determined in relation to the second risk index in such a way as, for example, “the second risk index is equal to or more than a second threshold value (e.g., ‘0.3’)”.

The third criterion is a criterion for discriminating an age group in which a risk that an infection disease diffuses from a target place to the residential area Ai of a visitor is high. The third criterion is previously determined in relation to the third risk index in such a way as, for example, “being an age group accounting for 90% of a total number of visitors”.

The risk index memory unit 105 is a memory unit that stores the first risk index R1, second risk index R2, and third risk index R3 for each target place. The first risk index R1, the second risk index R2, and the third risk index R3 are stored in the risk index memory unit 105 by the estimation unit 104.

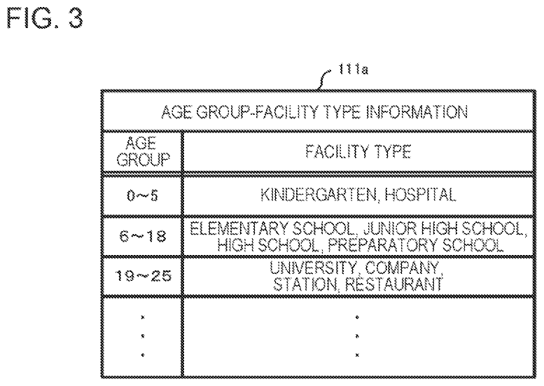

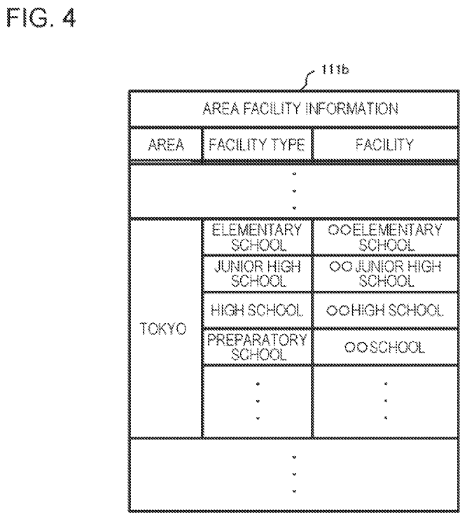

The facility memory unit 106 is a memory unit that previously stores age group-facility information for associating an age group with a facility for each area. In the present example embodiment, the age group-facility information is composed of age group-facility type information 111a and areal facility information 111b, as illustrated in FIGS. 3 and 4.

FIG. 3 is a diagram illustrating one example of the age group-facility type information 111a according to the present example embodiment. In the age group-facility type information 111a, an age group is associated with a facility type indicating a type of a facility. The age group and the facility type in the age group-facility type information 111a may be associated with a facility type indicating a type of a facility generally frequently utilized by a person belonging to the age group.

FIG. 4 is a diagram illustrating one example of the areal facility information 111b. The areal facility information 111b indicates a facility for each facility type in each area. The areal facility information 111b illustrated in FIG. 4 includes, for example, “oo elementary school” as a facility of a facility type “elementary school” in “Tokyo”.

Note that, although FIG. 4 illustrates an example in which a facility is identified by a name thereof, a facility may be identified not only by a name thereof but also by an address, an appropriately assigned code, or the like. Moreover, the areal facility information 111b may not be stored in the facility memory unit 106, but may be acquired from another non-illustrated apparatus via a network, and age group-facility information in this case may be composed only the age group-facility type information 111a.

The output unit 107 outputs, based on the first risk index R1, output information for supporting infection spread prevention of an infection disease. For example, the output unit 107 may output output information by displaying the output information, or may output output information by transmitting the output information to a non-illustrated external apparatus via a network.

The output information includes, for example, target place risk information 112a, occurrence place information 112b, diffusion place information 112c, and caution facility information 112d.

The target place risk information 112a is information in which the first risk index R1 is associated with a target place.

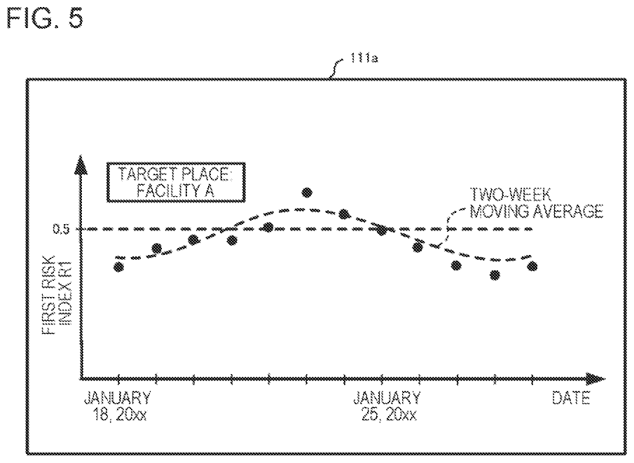

FIG. 5 illustrates one example of the target place risk information 112a. The target place risk information 112a illustrated in the figure includes the first risk index R1 for each day of a previously determined period and a two-week moving average thereof in a target place “facility A”.

Note that, although FIG. 5 illustrates an example in which a moving average is a two-week moving average, but a period for deriving a moving average is not limited to two weeks and may be changed as appropriate, and a moving average may not be included in the target place risk information 112a. Moreover, the target place risk information 112a may be information including the first risk index R1 of a specific day in a target place, and may further include a part or all of the visitor information 110.

The occurrence place information 112b is information relating to a target place where a risk of being infected with an infection disease is high. For example, the first risk index R1 of a target place is output when determined to satisfy the first criterion, and the occurrence place information 112b includes information for identifying a target place being associated with the first risk index R1satisfying the first criterion. The information for identifying a target place is, for example, a name of a facility being the target place, a location of the target place, or the like.

The diffusion place information 112c is information relating to a residential area of a visitor, and indicates the area Ai having a risk that an infection disease diffuses from a target place where a risk of being infected with the infection disease is high.

For example, the first risk index R1 is output when satisfying the first criterion, the second risk index R2 is output when satisfying the second criterion, and the diffusion place information 112c indicates the residential area Ai being associated with the second risk index R2 satisfying the second criterion.

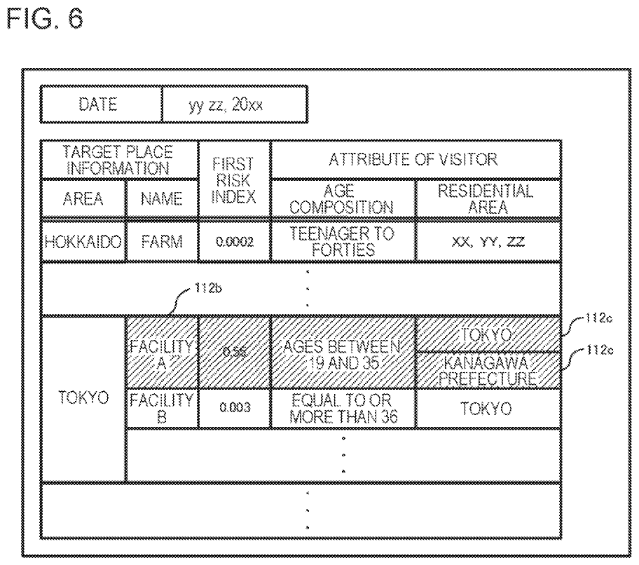

FIG. 6 illustrates one example of output information including the occurrence place information 112b and the diffusion place information 112c.

In the output information illustrated in FIG. 6, target place information of a date indicated by the date information, the first risk index R1, and an attribute of a visitor are associated.

The target place information includes an area where a target place is located, and a name thereof. The first risk index R1 is the first risk index R1 for the target place identified by the target place information. In the example illustrated in FIG. 6, the target place “facility A” being associated with the first risk index R1 satisfying a first criterion (e.g., being equal to or more than the first threshold value “0.5”) is indicated by hatching. Thus, FIG. 6 illustrates an example in which the occurrence place information 112b is indicated by indicating the target place being associated with the first risk index R1 satisfying the first criterion in a manner different from another target place.

An attribute of a visitor includes an age composition and the residential area Ai of the visitor coming to a target place identified by target place information.

The residential area Ai of the output information illustrated in FIG. 6 is a residential area where the first risk index R1 satisfies the first criterion and the second risk index R2 satisfies the second criterion. In the example illustrated in FIG. 6, the residential areas “Tokyo” and “Kanagawa Prefecture” where the first risk index R1 satisfies the first criterion and the second risk index R2 satisfies the second criterion are indicated by hatching. Thus, FIG. 6 illustrates an example in which a residential area where the first risk index R1 satisfies the first criterion and the second risk index R2 satisfies the second criterion is illustrated in a manner different from another residential area, and, thereby, the diffusion place information 112c is illustrated.

An age composition of the output information illustrated in FIG. 6 indicates an age group in which the first risk index R1 satisfies the first criterion, the second risk index R2 satisfies the second criterion, and the third risk index R3 satisfying the third criterion by the third risk index R3 satisfies the third criterion.

Note that, the diffusion place information 112c may be output when it is discriminated that the first risk index R1 of the target place satisfies the first criterion, and, in this case, the diffusion place information 112c indicating the residential areas Ai of all visitors may be output based on visitor information of a facility being associated with the first risk index R1 satisfying the first criterion.

The caution facility information 112d is information relating to a facility being related to an age group of a visitor among facilities provided in the residential area Ai of the visitor. The caution facility information 112d indicates a facility where there is a risk that an infection disease diffuses from a target place with a high risk of being infected with the infection disease.

For example, the first risk index R1 is output when satisfying the first criterion, the second risk index R2 is output when satisfying the second criterion, and the third risk index R3 is output when satisfying the third criterion. The caution facility information 112d in this case indicates a facility being associated with an age group of a visitor satisfying the third criterion among facilities provided in a residential area being associated with the second risk index R2 satisfying the second criterion.

More specifically, for example, among facilities provided in a residential area being associated with the second risk index R2 satisfying the second criterion, a facility being associated with an age group of a visitor satisfying the third criterion is identified by the following method.

The output unit 107 determines a facility type being associated with an age group of a visitor satisfying the third criterion in the age group-facility type information 111a. Then, the output unit 107 determines a facility being associated with the determined facility type among facilities provided in the residential area being associated with the second risk index R2 satisfying the second criterion in the areal facility information 111b. The output unit 107 outputs the caution facility information 112d including the determined facility.

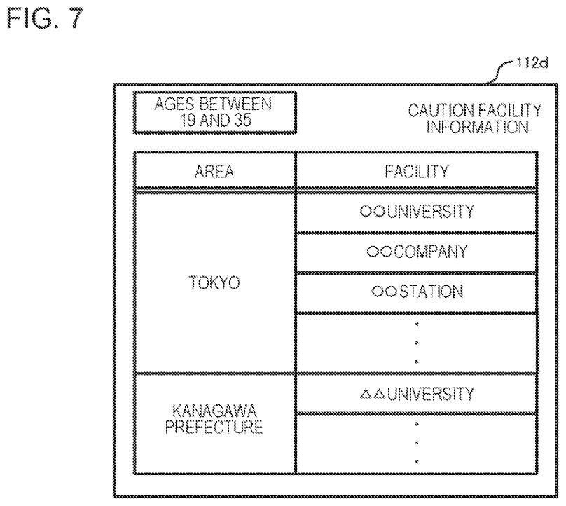

FIG. 7 illustrates one example of the caution facility information 112d. The caution facility information 112d in FIG. 7 indicates names of facilities in residential areas “Tokyo” and “Kanagawa” being associated with an age group “19 to 35”. Note that, a facility included in the caution facility information 112d is not limited to a name, and may include information such as an address with which a facility can be identified.

Note that, when the first risk index R1 satisfies the first criterion and the second risk index R2 satisfies the second criterion, caution facility information indicating all facilities being related to an age group of a visitor may be output among facilities provided in a residential area being associated with the second risk index R2 satisfying the second criterion.

### Physical Configuration of Infection Risk Estimation Apparatus 100

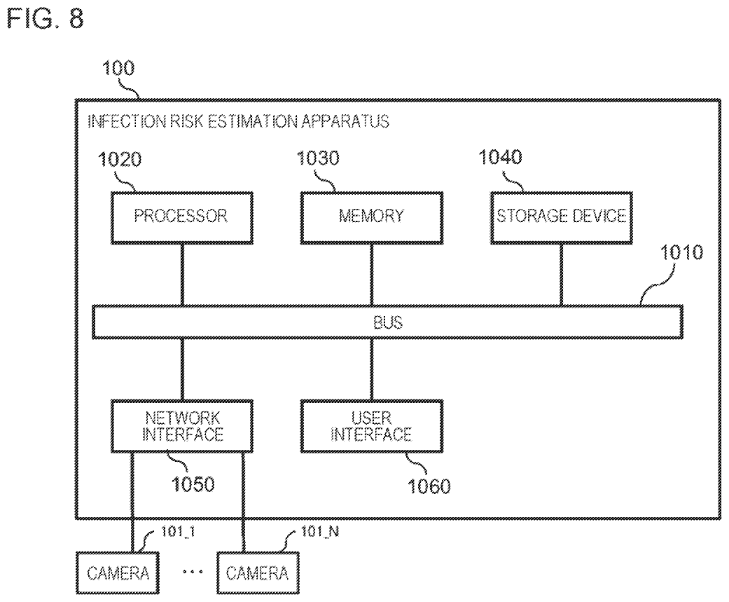

The infection risk estimation apparatus 100 is physically achieved by, for example, a general-purpose computer, and includes a bus 1010, a processor 1020, a memory 1030, a storage device 1040, a network interface 1050, and a user interface 1060, as illustrated in FIG. 8.

The bus 1010 is a data transmission path through which the processor 1020, the memory 1030, the storage device 1040, the user interface 1050, and the network interface 1060 transmit/receive data to/from each other. However, a method of mutually connecting the processor 1020 and the like is not limited to bus connection.

The processor 1020 is a processor achieved by a central processing unit (CPU), a graphics processing unit (GPU), or the like.

The memory 1030 is a main memory apparatus achieved by a random access memory (RAM) or the like.

The storage device 1040 is an auxiliary memory apparatus achieved by a hard disk drive (HDD), a solid state drive (SSD), a memory card, a read only memory (ROM), or the like. The storage device 1040 stores a program module for achieving each function of the contract terminal 101. The processor 1020 reads each of the program modules onto the memory 1030, executes the read program module, and thereby achieves each function being associated with the program module.

The user interface 1050 is a touch panel, a keyboard, a mouse, or the like as an interface for a user to input information, and a liquid crystal panel or the like as an interface for presenting information to a user.

The network interface 1060 is an interface for connecting the infection risk estimation apparatus 100 to a network N. One or a plurality of cameras 101_1 to 101_N are connected to the network interface 1060 via a network.

### Operation of Infection Risk Estimation Apparatus 100

From now on, an operation of the infection risk estimation apparatus 100 according to the present example embodiment is described with reference to the drawings.

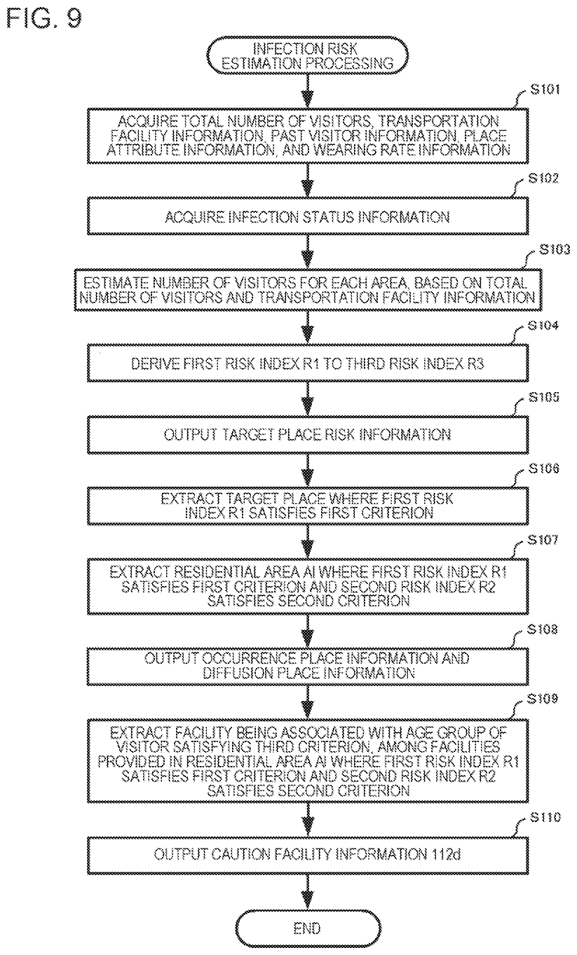

FIG. 9 is a flowchart illustrating one example of infection risk estimation processing according to the present example embodiment. The infection risk estimation processing is processing of supporting spread prevention of an infection disease by acquiring the first risk index R1 indicating a degree of a risk of being infected with the infection disease in a plurality of target places. The infection risk estimation processing may be automatically started, for example, at a previously determined cycle (e.g., one day), or may be started in response to an instruction of a user.

The first acquisition unit 102a acquires a total number of visitors to a target place, transportation facility information, past visitor information, wearing rate information, and place attribute information (step S101).

Transportation facility information acquired in step S101 includes image information generated by the camera 101 installed in a parking lot, and visitor image information generated by the camera 101 installed in a target place. Then, the first acquisition unit 102a acquires, based on visitor image information, wearing rate information regarding a visitor in a target place.

The second acquisition unit 102b acquires infection status information including the infection status of an infection disease in each of the areas Ai (step S102).

The first acquisition unit 102a estimates the number of visitors for each residential area, based on a total number of visitors to a target place acquired in step S101, and transportation facility information (step S103).

The transportation facility information used in step S103 is the acquired image information generated by the camera 101 installed in the parking lot. One example of a method of estimating the number of visitors for each residential area in step S103 is described below.

### Example of for Estimation Method of the Number of Visitors for Each Residential Area

A license plate includes a place name indicating a base of use and a classification number indicating the use. The first acquisition unit 102a determines the place name and classification number included in the license plate by performing image processing on an image including the license plate. A conventional image processing technique using pattern matching, machine learning, or the like may be utilized for the image processing.

The first acquisition unit 102a acquires the determined place name as the residential area Ai of the visitor. Moreover, the first acquisition unit 102a determines, according to the determined classification number, whether a car in the image is a bus or a passenger car other than a bus.

Then, the first acquisition unit 102a multiplies each of a number BNi of cars determined as buses and a number PNi of cars determined as passenger cars by previously determined predefined values PD1 and PD2 for each of the residential areas Ai.

The first acquisition unit 102a derives a weight Gi for each of the residential areas Ai, based on the numbers BNi and PNi of buses and passenger cars for each of the residential areas Ai, and the predefined values PD1 and PD2 for buses and passenger cars. The weight Gi of the residential area Ai is derived by, for example, an equation of “number BNi of buses×predefined value PD1 for bus+number PNi of passenger cars×predefined value PD1 for passenger car”.

An example of a target place “facility A” situated in Tokyo is explained. It is assumed that the number BNi of buses is two and the number PNi of passenger cars is one regarding the residential area “Tokyo” of “facility A”. It is assumed that the number PNi of passenger cars is two regarding the residential area “Kanagawa”. It is assumed that the number BNi of buses is one regarding the residential area “Shizuoka”.

Moreover, for example, it is assumed that the predefined value PD1 for a bus is “10”, 5 and the predefined value PD2 for a passenger car is “2”.

In this case, regarding the target place “facility A”, a weight of the residential area “Tokyo” is 22, a weight of the residential area “Kanagawa Prefecture” is 4, and a weight of the residential area “Shizuoka Prefecture” is 10.

The first acquisition unit 102a determines, based on a previously determined condition, whether there are many visitors with a transportation means other than a car, and acquires an estimate value of the number of visitors for each area by a method according to a result of determination.

The condition herein is that a total number of visitors is equal to or more than a value acquired by multiplying a sum total SN of cars (=sum total SBN of buses+sum total SPN of passenger cars) by a previously determined predetermined value PV.

In the example described above, the sum total SN is 6 in the target place “facility A”, and, therefore, when the predetermined value PV is “100”, a condition is that a total number of visitors is more than 600.

When the total number of visitors is less than a value acquired by multiplying the sum total SN by the predetermined value PV (i.e., when the condition described above is not satisfied), the first acquisition unit 102a acquires an estimate value of the number of visitors for each of the residential areas Ai by Equation (2).

[Mathematical 2]

Number of visitors to area Ai=total number of visitors×Gi/ΣGi Equation (2)

For example, in the example described above, when a total number of visitors to the target place “facility A” is 100, the number of visitors from Tokyo is estimated to be 61 (=100×22/(22+4+10)). Similarly, the number of visitors from Kanagawa Prefecture is estimated to be 11, and the number of visitors from Shizuoka Prefecture is estimated to be 28.

In other words, assuming that all the visitors have visited the target place by car, an estimate number of visitors for each of the residential areas Ai can be derived by proportionally dividing the total number of visitors by the weight Gi.

When a total number of visitors is equal to or more than a value acquired by multiplying the sum total SN of cars by the predetermined value PV, an estimate value of the number of visitors to the area Ai is acquired by use of different Equations (3) and (4) according to whether a target place belongs to the area Ai.

More specifically, when the total number of visitors is equal to or more than a value acquired by multiplying the sum total SN of cars by the predetermined value PV, the first acquisition unit 102a acquires, by Equation (3), an estimate value of the number of visitors to the area Ai other than an area At to which the target place belongs.

[Mathematical 3]

Number of visitors to area Ai (excluding area At.)=sum total SN×predetermined value PV×Gi/ΣGi Equation (3)

For example, in the example described above, when the total number of visitors to the target place “facility A” is 10600, the number of visitors from Kanagawa Prefecture is estimated to be 67 (=100×6×4/(22+4+10)). Similarly, the number of visitors from Shizuoka Prefecture is estimated to be 167.

In other words, it is assumed that the number of visitors by car is a number being equivalent to a value acquired by multiplying the sum total SN by the predetermined value PV, and all visitors from the area Ai (excluding the area At.) have visited the target place by car. Under such assumption, an estimate value of the number of visitors to the area Ai other than the area At can be derived by proportionally dividing the number of visitors by car with the weight Gi.

Further, when the total number of visitors is equal to or more than a value acquired by multiplying the sum total SN of cars by the predetermined value PV, the first acquisition unit 102a acquires, by Equation (4), an estimate value of the number of visitors to the area At to which the target place belongs.

[Mathematical 4]

Number of visitors to area Ai=(total number of visitors−sum total SN×predetermined value PV)+sum total SN×predetermined value PV×Gi/ΣGi Equation (4)

For example, in the above example, when a total number of visitors to the target place “facility A” is 10600, the number of visitors from Tokyo is estimated to be 100366 (=10600−100×6)+100×6×22/(22+4+10)).

In other words, it is assumed that the number of visitors by car is a number being equivalent to a value acquired by multiplying the sum total SN by the predetermined value PV, and all visitors other than a visitor by an automatic person are visitors from the area At. Under such assumption, an estimate value of the number of visitors to the area At is derived by adding the number of visitors other than a visitor by an automatic person, to the number of persons acquired by proportionally dividing the number of visitors by car by the weight Gi.

Note that, an acquisition method of the number of visitors for each residential area described herein is merely one example, and may be changed as appropriate. For example, the number of visitors for each residential area may be acquired by deriving a weight GI of each area, based on the number of visitors for each residential area included in the past visitor information in the target place, and residential area composition of a visitor acquired by a questionnaire to the visitor, and proportionally dividing the total number of visitors by the weight Gi.

The estimation unit 104 derives the first risk index R1 to third risk index R3, based on the information acquired in steps S101 to S103 (step S104). The estimation unit 104 stores the acquired first risk index R1 to third risk index R3 in the risk index memory unit 105.

The first risk index R1 is derived by the above Equation (1). The number of visitors to the area Ai is the estimate value acquired in step S103. An infection rate of the area Ai is an infection rate included in the infection status information acquired in step S103.

A coefficient p is a coefficient according to whether a target place included in the place attribute information acquired in step S101 is an outdoor facility clothing or an indoor facility. A coefficient q is a coefficient according to density of a person in a target place included in the place attribute information acquired in step S101. The coefficient p according to whether the target place is an outdoor facility clothing or an indoor facility and the coefficient q according to density of a person in the target place may be previously retained in, for example, the estimation unit 104.

The coefficient r is a coefficient according to wearing rate information acquired in step S101. For example, the estimation unit 104 may previously retain the coefficient r according to each of levels of wearing rates divided into 0 to 50%, 50% to 80%, 80% to 100%, and the like.

The second risk index R2 is, for example, a percentage of the number of visitors for each residential area estimated in step S103, and is derived by dividing visitors for each residential area by the total number of visitors.

The third risk index R3 is, for example, a percentage of a visitor for each age group included in past visitor information acquired in step S101, and is acquired based on the past visitor information.

The output unit 107 outputs target place risk information 11a (step S105).

Specifically, the output unit 107 acquires, from the risk index memory unit 105, the first risk index R1 for a period previously determined or indicated by a user, and derives two-week movement for the first risk index R1. Thereby, the output unit 107 generates and outputs the target place risk information 11a including the first risk index R1 for each day and the two-week movement average of the first risk index R1, for example, as illustrated in FIG. 5.

By referring to the target place risk information 112a, the user can easily recognize a risk of being infected with an infection disease in a target place in a period previously determined or indicated by the user. Therefore, it becomes possible to support an appropriate measure for preventing an infection spread of an infection disease.

Again, FIG. 9 is referred to.

The output unit 107 extracts a target place where the first risk index R1 satisfies the first criterion (step S106).

The output unit 107 extracts the residential area Ai where the first risk index R1 satisfies the first criterion and the second risk index R2 satisfies the second criterion (step S107).

The output unit 107 outputs the occurrence place information 112b including the target place extracted in step S106, and the diffusion place information 112c including the residential area Ai extracted in step S107 (step S108).

FIG. 6 is an example of output information including the occurrence place information 112b and the diffusion place information 112c output from the output unit 107 in step S108. In FIG. 6, the target place extracted in step S106 and the residential area Ai extracted in step S107 are indicated by hatching.

Note that, in the example embodiment, an example in which the occurrence place information 112b and the diffusion place information 112c are included in one piece of output information is illustrated, but the occurrence place information 112b and the diffusion place information 112c may be output from the output unit 107 as individual output information.

By referring to the occurrence place information 112b, the user can easily recognize, as an occurrence place, a target place where a risk of being infected with an infection disease is high. Therefore, it becomes possible to support an appropriate measure for preventing an infection spread of an infection disease.

By referring to the diffusion place information 112c, the user can easily recognize, as a diffusion place, the area Ai where a possibility that an infection disease diffuses from an occurrence place is high. Therefore, it becomes possible to support an appropriate measure for preventing an infection spread of an infection disease.

Again, FIG. 9 is referred to.

The output unit 107 extracts a facility being associated with an age group of a visitor satisfying the third criterion, among facilities provided in the residential area Ai where the first risk index R1 satisfies the first criterion and the second risk index R2 satisfies the second criterion (step S109).

The output unit 107 outputs the caution facility information 112d indicating the facility extracted in step S109 (step S110).

FIG. 7 is an example of the caution facility information 112d output from the output unit 107 in step S110. By referring to such caution facility information 112d, the user can easily recognize, as a caution facility, a facility where a possibility that an infection disease diffuses from an occurrence place is high. Therefore, it becomes possible to support an appropriate measure for preventing an infection spread of an infection disease.

Note that, in step S110, the output unit 107 may output caution facility information by transmitting the caution facility information to an apparatus (not illustrated) installed in a facility indicated by the caution facility information. Thereby, an employee or the like in a facility can learn that the facility is high in an infection risk of an infection disease, and take an appropriate measure. Therefore, it becomes possible to support an appropriate measure for preventing an infection spread of an infection disease.

So far, the first example embodiment according to the present invention has been described.

According to the present example embodiment, the infection risk estimation apparatus 100 acquires the first risk index R1 indicating a degree of a risk of being infected with an infection disease in a target place, by use of the visitor information 110 including an attribute of a visitor to a target place, and infection status information including infection status of an infection disease in each area.

By referring to the first risk index R1, a user can easily learn a target place where an infection risk is high. Moreover, by transmitting the first risk index R1 to a previously determined apparatus or the like, information of a target place where an infection risk is high can be provided. Thereby, a measure for preventing an infection spread can be taken in such a way that a visitor to a target place refrains from going out and a person concerned in the target place calls attention. Therefore, it becomes possible to support an action for preventing the infection spread.

The present invention is not limited to the first example embodiment described above, and may be modified as follows.

### Modified Example 1: Modified Example of Acquisition Method of the Number of Visitors For Each Residential Area

In the example embodiment, an example in which the number of visitors for each residential area is acquired based on passage facility information has been described, but a method of acquiring the number of visitors for each residential area is not limited thereto. For example, the number of visitors for each residential area may be acquired based on movement route information, guest information, and the like.

Movement route information is information including a movement route by a movement means for a visitor to visit a target place. As an example of a movement means, a railroad, a car, and a bicycle can be cited.

Movement route information of a railroad includes a boarding station and a getting-off station of a person leaving from a station. Movement route information of a railroad is acquired by an automatic ticket gate at a station, for example, when electronic payment is made during leaving by utilizing the automatic ticket gate, and is acquired by a first acquisition unit 102a via a server apparatus (not illustrated) of a railroad institution or the like.

In this case, when a getting-off station included in the movement route information is within a previously determined distance from a target place, the first acquisition unit 102a acquires the movement route information as movement route information of a visitor to the target place. The first acquisition unit 102a acquires, as the residential area Ai of a visitor, the area Ai where a boarding station included in movement route information is provided.

Movement route information of a car includes a movement route from a departure point to a destination point of the car. Movement route information of a car is acquired by a global positioning system (GPS) apparatus mounted on the car or a control apparatus that controls autonomous driving, and is acquired by the first acquisition unit 102a from the apparatuses (not illustrated) via a network.

In this case, when a destination point included in the movement route information is a parking lot being associated with a target place by the association condition described above, the first acquisition unit 102a acquires the movement route information as movement route information of the visitor to the target place. Moreover, the first acquisition unit 102a acquires, as a residential area of the visitor to the target place, an area including a departure point included in the movement route information.

Movement route information of a bicycle includes a movement route from a boarding base to a getting-off base. As to such movement route information, for example, in a case of a bicycle provided by a rental cycle, a bicycle parking facility where the bicycle is rented is a boarding base, and a bicycle parking facility where the bicycle is returned is a getting-off base. A rental cycle is a service of lending a bicycle that can be utilized between bicycle parking facilities provided at previously determined bases.

In this case, when a getting-off base included in movement route information is a bicycle parking facility within a previously determined range from a target place, the first acquisition unit 102a acquires the movement route information as movement route information of a visitor to the target place. Moreover, the first acquisition unit 102a acquires, as a residential area of the visitor to the target place, an area including a boarding base included in the movement route information.

Guest information is information relating to a guest in an accommodation facility, and includes, for example, a name or an address of the accommodation facility, and an address and an age of the guest, and the like.

For example, when an accommodation facility identified by a name or an address of the accommodation facility is within a previously determined distance from the target place, the first acquisition unit 102a acquires the guest information as guest information of a visitor to the target place. The first acquisition unit 102a acquires, as the residential area Ai of the visitor, an area including an address of the guest included in the guest information. Moreover, the first acquisition unit 102a acquires the age included in the guest information, as an age group of the visitor. Note that, the residential area Ai may include nationality.

The first acquisition unit 102a may acquire at least one of movement history information of at least one transportation means of a railroad, a car, and a bicycle, and guest information, in addition to transportation facility information according to the example embodiment, or instead of the transportation facility information. Then, the first acquisition unit 102a may acquire the number of visitors for each residential area, based on one of a boarding station, a departure point, and a boarding base in the acquired movement history information. When movement history information of a car is acquired, transportation facility information according to the example embodiment may not be acquired.

### Modified Example 2: Modified Example of An Acquisition Method of An Age Group of a Visitor

For example, a first acquisition unit 102a may acquire an age group of a visitor to a target place by acquiring visitor image information from a camera 101 installed in the target place, and performing image processing on the visitor image information. Moreover, for example, the first acquisition unit 102a may acquire an age range of a visitor to a target place by acquiring image information further including a passenger of a car from the camera 101 installed in a parking lot, and performing image processing on the image information. The image processing applied therein may be a conventional image processing technique, and uses, for example, a learned learning model that inputs an image of a person and outputs an age group of the person.

### Modified Example 3: Modified Example That Does Not Require the Total Number of Visitors

In the example embodiment, an example in which the number of visitors for each residential area Ai is estimated by use of different Equation (2) or Equations (3) and (4) according to whether the total number of visitors is less than a value acquired by multiplying a sum total SN of cars by a previously determined predetermined value PV has been described. However, without using the total number of visitors, a weight Gi of all residential areas may be adopted as the number of visitors for each of the residential areas Ai.

According to the present modified example, even when the total number of visitors cannot be acquired, risk indices R1 to R3 can be derived. Particularly, it is effective when almost all visitors visit a target place by car. cl Modified Example 4: First Example of Vaccination Status Information and Negative Status Information

The first acquisition unit 102a may further acquire at least one of vaccination status information and negative status information.

The vaccination status information is information indicating a degree of persons vaccinated with vaccination against an infection disease, and indicates, for example, a number N1 of persons vaccinated against an infection disease in a target place. The negative status information is information indicating a degree of a person who has acquired a negative certificate for an infection disease, and indicates, for example, a number N2 of persons who have acquired a negative certificate for an infection disease in a target place.

In this case, for example, the number of visitors to an area Ai can be derived by Equation (5) replacing Equation (2) and Equation (6) replacing Equation (4). In other words, in Equations (5) and (6), “total number of visitors−number of persons N1−number of persons N2” is adopted instead of “total number of visitors” in each of Equations (2) and (4) according to the example embodiment. A vaccinated person and a person who has acquired a negative certificate are generally considered to have a low infection risk, and the number of visitors to area Ai according to the modified example is the number of visitors for each area regarding a person having equal to or more than a certain degree of an infection risk.

[Mathematical 5]

Number of visitors to area Ai=(total number of visitors−number of persons N1−number of persons N2)×Gi/ΣGi Equation (5)

[Mathematical 6]

Number of visitors to area Ai=(total number of visitors−number of persons N1−number of persons N2−sum total SN×predetermined value PV)+sum total SN×predetermined value PV×Gi/ΣGi Equation (6)

By deriving a first risk index R1 by use of the number of visitors for each area regarding a person having equal to or more than a certain degree of an infection risk, a more appropriate index indicating a degree of a risk of being infected with an infection disease can be acquired. Therefore, it becomes possible to support an action for preventing an infection spread.

### Modified Example 5: First Modified Example of Equation (1) for Deriving a First Risk Index R1)

Vaccination status information may be information indicating a degree of a person vaccinated with vaccination against an infection disease, and is not limited to information indicating the number of vaccinated persons. For example, the vaccination status information may be information indicating a nationwide percentage of vaccinated persons, or may be information indicating a percentage of vaccinated persons in an area including a target place.

Negative status information may be information indicating a degree of a person who has acquired a negative certificate of an infection disease, and is not limited to information indicating the number of persons who have acquired a negative certificate. The negative status information may be, for example, information indicating a nationwide percentage of a person who has acquired a negative certificate, or may be information indicating a percentage of a person who has acquired negative certificate in an area including a target place.

Then, the first risk index R1 may be derived by multiplying either or both of coefficients s and t according to each of vaccination status information and negative status information, by a value acquired by adding up 47 prefectures instead of coefficients p to r or in addition thereto.

For example, a value appropriately determined according to vaccination status information is set for the coefficient s. Generally, infection may be suppressed by vaccination against an infection disease. In such a case, a previously determined value may be set for the coefficient s in such a way as to become smaller as a value of vaccination status information is greater.

For example, a value appropriately determined according to negative situation information is set for the coefficient t. Generally, it is considered to be difficult be infected with an infection disease as a percentage of negative persons is larger. In such a case, a previously determined value may be set for the coefficient tin such a way as to become smaller as a value of the negative status information is greater.

### Modified Example 6: Second Modified Example of Equation (1) for Deriving First Risk Index R1)

A part (one or a plurality) of coefficients p to t may be adopted for deriving the first risk index R1, and the coefficients p to t may not be adopted for deriving the first risk index R1.

However, by using a part or all of the coefficients p to tin order to acquire the first risk index R1, a more appropriate index indicating a degree of a risk of being infected with an infection disease can be acquired. Therefore, it becomes possible to support an action for preventing an infection spread.

### Modified Example 7: Modified Example of Indices R1 to R3)

In the present example embodiment, an example in which almost continuous numerical values are adopted as the indices R1 to R3 has been described. However, the indices R1 to R3 may be indices (numerical values, alphabets, or the like) indicating, in stages, a risk or a degree of the number. The indices R1 to R3 are not limited to values derived every day, and may be acquired for each previously determined period, or may be an average value or the like for a previously determined period.

### Second Example Embodiment

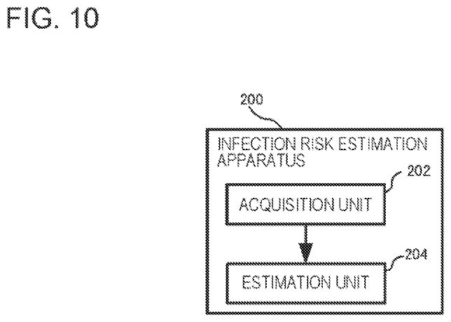

An infection risk estimation apparatus 200 according to a second example embodiment of the present invention functionally includes an acquisition unit 202 and an estimation unit 204, as illustrated in FIG. 10.

The acquisition unit 202 acquires visitor information and infection status information.

The visitor information is information including an attribute of a visitor to a target place, similarly to the first example embodiment. Moreover, the infection status information is also information including infection status of an infection disease in each area Ai, similarly to the first example embodiment.

The visitor information according to the present example embodiment includes, for example, a residential area Ai of a visitor as an attribute of the visitor, and the acquisition unit 202 may acquire visitor information by an appropriate method.

Specifically, for example, the acquisition unit 202 may acquire visitor information, based on visitor image information from a camera (not illustrated), similarly to the first example embodiment. Moreover, for example, the acquisition unit 202 may acquire visitor information by input of a user. Further, for example, the acquisition unit 202 may acquire visitor information from an external apparatus (not illustrated) via a network N or the like.

The estimation unit 204 acquires the first risk index R1 by use of visitor information and infection status information acquired by the acquisition unit 202. The first risk index R1 is an index indicating a degree of a risk of being infected with an infection disease in a target place, similarly to the first example embodiment.

The first risk index R1 according to the present example embodiment is derived by deriving a product of the number of visitors and an infection rate for each of the residential areas Ai, and adding up the product regarding all of the residential areas Ai, for example, as illustrated in Equation (7).

[Mathematical 7]

First risk index R1=Σ(number of visitors to area Ai×infection rate in area Ai) Equation (7)

The infection risk estimation apparatus 200 according to the second example embodiment may be physically configured similarly to the infection risk estimation apparatus 100 according to the first example embodiment (refer to FIG. 8).

From now on, an operation of the infection risk estimation apparatus 200 according to the present example embodiment is described with reference to the drawings.

The acquisition unit 202 acquires visitor information and infection status information (step S201).

The estimation unit 204 acquires the first risk index R1, based on the information acquired in step S201 (step S202).

Specifically, for example, the estimation unit 204 acquires the number of visitors to the area Ai, based on the visitor information acquired in step S201. Moreover, the estimation unit 204 acquires infection status of an infection disease in the area Ai, based on the infection status information acquired in step S201. Then, the first risk index R1 of a target place is acquired by applying the acquired values to Equation (1).

According to the present example embodiment as well, similarly to the first example embodiment, the infection risk estimation apparatus 200 acquires the first risk index R1 indicating a degree of a risk of being infected with an infection disease in a target place, by use of visitor information including an attribute of a visitor to the target place, and infection status information including infection status of an infection disease in each area.

Thereby, similarly to the first example embodiment, a user can easily learn, by referring to the first risk index R1, a target place where an infection risk is high. Moreover, information of a target place where an infection risk is high can be provided by transmitting the first risk index to a previously determined apparatus or the like. Thereby, a measure for preventing an infection spread can be taken in such a way that a visitor to a target place refrains from going out and a person concerned in the target place calls attention. Therefore, it becomes possible to support an action for preventing the infection spread.

The example embodiments and modified examples of the present invention have been described above with reference to the drawings, but are exemplifications of the present invention, and various configurations other than those described above can also be adopted.

For example, although a plurality of processes (pieces of processing) are described in order in a plurality of flowcharts used in the above description, an execution order of the processes executed in each of the example embodiments is not limited to the described order. In each of the example embodiments, an order of the illustrated processes can be changed to an extent that causes no problem in terms of content. Moreover, the example embodiments and modified examples described above can be combined to an extent that content does not contradict.

Some or all of the above-described example embodiments can also be described as, but are not limited to, the following supplementary notes.

1. An infection risk estimation apparatus including:
an acquisition means for acquiring visitor information including an attribute of a visitor to a target place, and infection status information including infection status of an infection disease in each area; and
an estimation means for acquiring, by use of the visitor information and the infection status information, a first risk index indicating a degree of a risk of being infected with the infection disease in the target place.
   2. The infection risk estimation apparatus according to supplementary note 1, wherein an attribute of the visitor includes a residential area of the visitor.
   3. The infection risk estimation apparatus according to supplementary note 1 or 2, further including
an output means for outputting, based on the first risk index, output information for supporting infection spread prevention of the infection disease.
   4. The infection risk estimation apparatus according to supplementary note 3, wherein
the output means outputs, as the output information, target place risk information in which the first risk index is associated with the target place.
   5. The infection risk estimation apparatus according to supplementary note 3 or 4, wherein
the output means outputs, as the output information, occurrence place information relating to the target place where a risk of being infected with the infection disease is high, when the first risk index satisfies a first criterion.
   6. The infection risk estimation apparatus according to any one of supplementary notes 3 to 5, wherein
the output means outputs, as the output information, diffusion place information relating to a residential area of the visitor, based on the visitor information, when the first risk index satisfies a first criterion.
   7. The infection risk estimation apparatus according to any one of supplementary notes 3 to 6, wherein
the estimation means further acquires, by use of the first risk index and the visitor information, a second risk index being an index according to a number of visitors for each residential area.
   8. The infection risk estimation apparatus according to supplementary note 7, wherein
when the first risk index satisfies the first criterion and the second risk index satisfies a second criterion, the output means outputs, as the output information, diffusion place information relating to a residential area being associated with a second risk index satisfying the second criterion.
   9. The infection risk estimation apparatus according to supplementary note 7 or 8, wherein
an attribute of the visitor further includes an age group of the visitor, and,
when the first risk index satisfies the first criterion and the second risk index satisfies a second criterion, the output means outputs, as the output information, caution facility information relating to a facility being associated with an age group of the visitor among facilities provided in a residential area being associated with a second risk index satisfying the second criterion.
   10. The infection risk estimation apparatus according to supplementary note 9, wherein
the estimation means further acquires a third risk index being an index according to a number of visitors for each age group, by use of the first risk index and the visitor information,
when the first risk index satisfies the first criterion, the second risk index satisfies a second criterion, and the third risk index satisfies a third criterion, the output means outputs, as the output information, caution facility information relating to a facility being associated with an age group of the visitor satisfying the third criterion among facilities provided in a residential area being associated with a second risk index satisfying the second criterion.
   11. The infection risk estimation apparatus according to any one of supplementary notes 1 to 10, wherein
the infection status information includes an infection rate of an infection disease in each of the areas, as infection status of the infection disease in each of the areas.
   12. The infection risk estimation apparatus according to any one of supplementary notes 1 to 11, wherein
the acquisition means further acquires a wearing rate indicating a percentage of a person wearing a cover covering a mouth among the visitors, and
the estimation means derives the first risk index further by use of the wearing rate.
   13. The infection risk estimation apparatus according to supplementary note 12, wherein the acquisition means acquires the wearing rate, based on an image captured by a capturing means provided in the target place.
   14. The infection risk estimation apparatus according to any one of supplementary notes 1 to 13, wherein
the acquisition means further acquires at least one piece of information of place attribute information indicating an attribute of the target place, vaccination status information indicating a degree of a person vaccinated with vaccination against the infection disease, and negative status information indicating a degree of a person who has acquired a negative certificate for the infection disease, and
the risk estimation means derives the first risk index further by use of the at least one piece of information.
   15. The infection risk estimation apparatus according to any one of supplementary notes 1 to 14, wherein
the acquisition means acquires a residential area included in the visitor information by use of at least one of transportation facility information acquired in a transportation facility previously associated with the target place, movement route information including a movement route by a movement means for the visitor to visit the target place, past visitor information in the target place, and guest information in an accommodation facility within a previously determined distance from the target place.
   16. The infection risk estimation apparatus according to supplementary note 15, wherein the transportation facility information includes an image of a license plate of a car captured by an image capturing means provided in a parking lot as the transportation facility.
   17. An infection risk estimation method including, by a computer:
acquiring visitor information including an attribute of a visitor to a target place, and infection status information including infection status of an infection disease in each area; and
acquiring, by use of the visitor information and the infection status information, a first risk index indicating a degree of a risk of being infected with the infection disease in the target place.
   18. A program for causing a computer to execute:
acquiring visitor information including an attribute of a visitor to a target place, and infection status information including infection status of an infection disease in each area; and
acquiring, by use of the visitor information and the infection status information, a first risk index indicating a degree of a risk of being infected with the infection disease in the target place.

This application is based upon and claims the benefit of priority from Japanese patent application No. 2021-050714, filed on Mar. 24, 2021, the disclosure of which is incorporated herein in its entirety by reference.

### REFERENCE SIGNS LIST

100, 200 Infection risk estimation apparatus
10 101, 101_1 to 101_N Camera
102, 202 Acquisition unit
102a First acquisition unit
102b Second acquisition unit
104 Estimation unit
105 Risk index memory unit
106 Facility memory unit
107 Output unit
110 Visitor information
111a Age group-facility type information
111b Areal facility information
112a Target place risk information
112b Occurrence place information
112c Diffusion place information
112d Caution facility information

What is claimed is:

1. An infection risk estimation apparatus comprising:
at least one memory configured to store instructions; and
at least one processor configured to execute the instructions to perform operations comprising:
acquiring visitor information including an attribute of a visitor to a target place, and infection status information including infection status of an infection disease in each area; and
acquiring, by use of the visitor information and the infection status information, a first risk index indicating a degree of a risk of being infected with the infection disease in the target place.

2. The infection risk estimation apparatus according to claim 1, wherein
the attribute of the visitor includes a residential area of the visitor.

3. The infection risk estimation apparatus according to claim 1, the operations further comprising
outputting, based on the first risk index, output information for supporting infection spread prevention of the infection disease.

4. The infection risk estimation apparatus according to claim 3, wherein
the output information includes target place risk information in which the first risk index is associated with the target place.

5. The infection risk estimation apparatus according to claim 3, wherein
the output information includes occurrence place information relating to the target place where a risk of being infected with the infection disease is high, when the first risk index satisfies a first criterion.

6. The infection risk estimation apparatus according to claim 3, wherein
the output information includes diffusion place information relating to a residential area of the visitor, based on the visitor information, when the first risk index satisfies a first criterion.

7. The infection risk estimation apparatus according to claim 3, the operations further comprising wherein
acquiring, by use of the first risk index and the visitor information, a second risk index being an index according to a number of visitors for each residential area, and, wherein
when the first risk index satisfies the first criterion and the second risk index satisfies a second criterion, the output information includes diffusion place information relating to a residential area being associated with a second risk index satisfying the second criterion.

8. The infection risk estimation apparatus according to claim 7, wherein
an attribute of the visitor further includes an age group of the visitor, and,
when the first risk index satisfies the first criterion and the second risk index satisfies a second criterion, the output information includes caution facility information relating to a facility being associated with an age group of the visitor, among facilities provided in a residential area being associated with a second risk index satisfying the second criterion.

9. The infection risk estimation apparatus according to claim 7, the operations further comprising
acquiring a third risk index being an index according to a number of visitors for each age group, by use of the first risk index and the visitor information, and wherein
when the first risk index satisfies the first criterion, the second risk index satisfies a second criterion, and the third risk index satisfies a third criterion, the output information includes caution facility information relating to a facility being associated with an age group of the visitor satisfying the third criterion, among facilities provided in a residential area being associated with a second risk index satisfying the second criterion.

10. The infection risk estimation apparatus according to claim 1, the operations further comprising
acquiring a wearing rate indicating a percentage of a person wearing a cover covering a mouth, among the visitors, and wherein
the first risk index is derived further by use of the wearing rate.

11. The infection risk estimation apparatus according to claim 1, the operations further comprising
acquiring at least one piece of information of place attribute information indicating an attribute of the target place, vaccination status information indicating a degree of a person vaccinated with vaccination against the infection disease, and negative status information indicating a degree of a person who has acquired a negative certificate for the infection disease, and wherein
first risk index is derived further by use of the at least one piece of information.

12. An infection risk estimation method comprising,
by at least one computer:
acquiring visitor information including an attribute of a visitor to a target place, and infection status information including infection status of an infection disease in each area; and
acquiring, by use of the visitor information and the infection status information, a first risk index indicating a degree of a risk of being infected with the infection disease in the target place.

13. A non-transitory storage medium storing a program for causing at least one computer to execute:
acquiring visitor information including an attribute of a visitor to a target place, and infection status information including infection status of an infection disease in each area; and
acquiring, by use of the visitor information and the infection status information, a first risk index indicating a degree of a risk of being infected with the infection disease in the target place.

14. The infection risk estimation method according to claim 12, wherein
the attribute of the visitor includes a residential area of the visitor.

15. The infection risk estimation method according to claim 12, further comprising
outputting, based on the first risk index, output information for supporting infection spread prevention of the infection disease.

16. The infection risk estimation method according to claim 15, wherein
the output information includes target place risk information in which the first risk index is associated with the target place.

17. The non-transitory storage medium storing the program for causing at least one computer according to claim 13, wherein
the attribute of the visitor includes a residential area of the visitor.

18. The non-transitory storage medium storing the program for causing at least one computer according to claim 13, further to execute
outputting, based on the first risk index, output information for supporting infection spread prevention of the infection disease.

19. The non-transitory storage medium storing the program for causing at least one computer according to claim 18, wherein
the output information includes target place risk information in which the first risk index is associated with the target place.
